(19) United States
(12) Patent Application Publication
(10) Pub. No.: US 2004/0197767 A1
(43) Pub. Date: Oct. 7, 2004

(54) Whole cell assay

(76) Inventors: Frank Fan, Madison, WI (US); Yinduo Ji, St. Paul, MN (US); Howard Kallender, Wayne, PA (US); Tong Li, Audubon, PA (US); Damien McDevitt, Berwyn, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/492,565

(22) PCT Filed: Oct. 15, 2002
(86) PCT No.: PCT/US02/32849

(30) Foreign Application Priority Data
Oct. 17, 2001 (US) 09/981,121

(51) Int. Cl. C12Q 1/00; C12Q 1/68

(52) U.S. Cl. 435/4; 435/6

(57) ABSTRACT

This invention relates to newly developed methods for discovering therapeutic compounds using a cell-based assay system. This invention also relates to compositions of matter useful in carrying out the methods of the invention as well as therapeutic compounds developed using such methods.

### FIELD OF THE INVENTION

This invention relates to newly developed methods for discovering a range of therapeutic compounds, particularly antimicrobial compounds, and identifying their cellular targets using a whole cell assay. It is particularly suited for carrying out therapeutic compound screening assays in bacterial host cells and eukaryotic host cells. This invention also relates to compositions of matter useful in carrying out the methods of the invention as well as therapeutic compounds developed using such methods.

### BACKGROUND OF THE INVENTION

There is a need for methods for screening for novel therapeutic compounds, such as the screening methods of the invention. Such methods have a present benefit of being useful to screen compounds for antibiotic activity that can play a role in preventing, ameliorating or correcting infections, dysfunctions or diseases, such as bacterial infections.

This technology is also particularly useful to identify target(s) of antimicrobial compounds, by looking for a modulation of a detectable signal with an increase in gene expression, such as, an increase in MIC when gene expression is increased.

Glossary

The following definitions are provided to facilitate understanding of certain terms used frequently herein. Certain other definitions are provided elsewhere herein.

“Host cell” is a cell which has been transformed or transfected or into which genetic information has been introduced, or which is capable of transformation or transfection or introduction into said cell by an exogenous polynucleotide sequence.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein.

“Polynucleotide(s)” generally refers to any polyribonucleotide or polydeoxribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotide's as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

“Therapeutic drugs” as the term is used herein, can be identified from candidate compounds that alter metabolism, for example, Potential therapeutic compounds identified using the method of the invention include, among other things, small organic molecules, polynucleotides, peptides, polypeptides and antibodies that bind host cell polynucleotides or polypeptides, or mimic the activity of a host cell polypeptides.

“Contacting said host cell in a second contacting step” as the term is used herein, is for one example, a same host cell could be contacted in two different contacting steps. Each contacting step can be at a different level of induction, (and the altered metabolism is detected twice). For example:

1. The first contacting step could include:

A host cell comprising at least one recombinant regulatable gene.

Contacting the host cell with at least one candidate compound and inducer at predetermined concentrations, for example, 1× and 0.5× respectively.

The host cell may have an altered metabolism that may be detected.

2. A second contacting step could include, for example:

Contacting the host cell of first contacting step with additional inducer to a predetermined level, for example 0.8×.

If the candidate compound is product-specific, the host cell from the first contacting step will express more gene product and result in a new altered metabolism that may be detected.

The aforementioned example could have any number of substitutions in the second contacting step, such as, increasing the candidate compound and/or inducer, decreasing the candidate compound and/or inducer, using the same host cell for the first and second contacting steps or using separate cultures of the same host cell. Tracking the concentrations and/or measurements of the variables are not issues.

### SUMMARY OF THE INVENTION

An object of the invention is a method of screening for therapeutic drugs comprising the steps of: providing at least one host cell comprising at least one recombinant regulatable gene; contacting said host cell with at least one candidate compound at least one level of induction of gene expression; and detecting altered metabolism in said host cell of the contacting step.

Another object of the invention is a method of screening for therapeutic drugs comprising the steps of: providing at least one host cell comprising at least one recombinant regulatable gene; contacting said host cell in a first contacting step with at least one candidate compound at a first level of induction of gene expression; detecting altered metabolism in said host cell of said first contacting step; contacting said host cell in a second contacting step with at least one candidate compound at a second level of induction of gene expression; and detecting altered metabolism in said host cell of said second contacting step.

A further embodiment of the invention is a method wherein a recombinant gene is on an episomal element or integrated into a chromosome of said host cell.

Another embodiment of the invention is a method wherein a recombinant antisense of a gene is on an episomal element of said host cell.

Another embodiment is a method wherein the at least one level is two or more levels.

A still further embodiment of the invention is a method wherein said at least one recombinant regulatable gene is selected from the group consisting of a Gram positive bacterium, a Gram negative bacterium, a streptococcus, S. pneumoniae, a staphylococcus, S. aureus, enterococci, Enterococcus faecalis, Enterococcus faecium, a Bacillus, and Bacillus subtilis.

Another embodiment of the invention is a method wherein said at least one recombinant regulatable gene is a human gene.

Further provided by the invention is a method of claim wherein said at least one recombinant regulatable gene is selected from the group consisting of hepatic cells, vascular cells, neuronal cells, dermal cells, renal cells, pancreatic cells, gut cells, bone cells, muscle cells, transformed cells, and carcinoma cells.

Another embodiment is a method wherein said altered metabolism comprises an alteration or modulation in viability, growth, proliferation, differentiation, gene expression, gene product activity, lysis, cell division, chemotaxis, motility, cytoskelatel structure or motion, nuclear structure, meiosis, mitosis, translation, transcription, sister chromatid exchange, cell permeability, surface receptors, refractive index, sporulation, tumbling in solution, optical density, protein folding, protein content, nucleic acid content, phagocytosis or protein stability.

A preferred embodiment of the invention is a method wherein said at least one recombinant regulatable gene is selected from the group consisting of a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus aureus strain RN4220, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, a unicellular or filamentous eukaryote, a protozoan, a fungus, a member of the genus Saccharomyces, Kluveromyces, or Candida, and a member of the species Saccharomyces ceriviseae, Kluveromyces lactis, or Candida albicans.

Another preferred embodiment of the invention is a method of screening for therapeutic drugs comprising the steps of: providing at least one host cell comprising at least one gene under the control of a regulatable promoter; contacting the host cell with at least one candidate compound; and detecting altered metabolism in the host cell of the contacting step.

Another embodiment of the invention is a method wherein the genes under the control of the regulatable promoter are on an episomal element or integrated into a chromosome of the host cell.

Yet another embodiment of the invention is a method wherein the gene expression level is regulated.

A method wherein the gene is selected from the group consisting of eubacteria or eukaryotes, particularly a human is also provided by the invention.

A further embodiment of the invention is a method wherein the altered metabolism comprises inhibition of the encoded protein activity.

A still further embodiment of the invention is a method wherein the detecting step further comprises detecting a toxic effect of inhibiting the encoded protein activity.

A method is also provided wherein the detecting step further comprises detecting host cell death.

A method is provided wherein the host cell lacks a complete copy of the gene under the control of its native promoter.

A method or composition wherein the regulated genes are on episomal element or integrated into a chromosome of the host cell is also provided by the invention.

A host cell wherein the gene expression level is regulated is also provided by the invention.

Also provided as an embodiment of the invention is a polynucleotide comprising a gene expressibly linked to an regulatable promoter.

### BRIEF DESCRIPTION OF THE DRAWINGS

FIG. 1 shows a graph demonstrating the putative mechanism of one preferred embodiment of the invention. Screen at least 2 levels of gene production and look for compounds that are more inhibitory at the lower level(s). These will be specific inhibitors against this gene product rather than general bacteria.

### DETAILED DESCRIPTION OF THE INVENTION

The natural promoter of the gene is replaced with a heterologous, regulatable promoter (e.g., an inducible or repressible promoter) in the chromosome of a gene-expressing microbial host cell, such as by homologous recombination (in a preferred embodiment insertional mutagenesis is used since, for example, it is more rapid than a double crossover and should give the same phenotype. Such a gene construct comprising a regulatable promoter is referred to herein as a “hybrid gene,” “regulatable gene” or “recombinant regulatable gene.” However, by using this method there is an extra copy of the first stretch of base pairs (e.g., about 300-700 base pairs, preferably about 500 base pairs) of the gene present, still under the control of the native promoter. This is, in a preferred embodiment, not sufficient sequence to encode active gene product.

Other embodiments of the invention include, for example, double crossover mutagenesis to provide efficient and complete promoter replacement; insertional mutagenesis to place a gene plus recombinant promoter in a specific, non-essential locus, and knocking out the chromosomal copy of the gene along with its natural promoter; transforming host cells with a plasmid containing the gene under control of the regulatable promoter; transforming host cells with a plasmid containing the gene under control of the regulatable promoter, and then knocking out the chromosomal copy of the gene plus the natural promoter; transforming host cells with a plasmid containing partial antisense strand of the gene under the control of the regulatable promoter.

Preferred host cells and other cells useful in the invention include, but are not limited to, any bacteria, and also a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, particularly Staphylococcus aureus strain RN4220, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (ii) an archaeon, including but not limited to Archaebacter, (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans, (iv) a eukaryotic cell, tissue, organ, or organisms, especially human cells and tissues, including but not limited to, hepatic cells, vascular cells, neuronal cells, dermal cells, renal cells, pancreatic cells, gut cells, bone cells, muscle cells, transformed cells, and carcinoma cells and (v) CHO, COS, HeLa, C127, 3T3, BHK, 293 and Bowes melanoma cells.

Regulatable promoters, particularly inducible promoters useful in the invention include, but are not limited to, P_{xylA} plus the xylR repressor gene, from various bacteria, such as Bacillus sp. and Lactobacillus pentosus; P_{lacA} plus the lacR or lacI repressor gene, from various bacteria, such as E. coli, S. aureus and Lactococcus lacti; hybrid promoters consisting of, for example, an E. coli lac repressor/operator and a −10 and −35 region of various promoters, such as phages SP0-1 (known as P_{spac}) and T5; P_{xyl/tet}—a hybrid consisting of the E. coli Tn10 tet repressor/operator and the Bacillus subtilis xylA −10 and −35 regions; P_{T7} plus the T7 RNA polymerase gene under the control of one of the described promoters; Pₜᵣₚ from various bacteria; φ31 middle promoter from Lactococcus lactis; Lantibiotic inducible promoters, such as P_{nisA} or P_{nisF} from Lactococcus lactis or P_{spaB} from Bacillus subtilis; and Galactose-inducible and Thiostrepton-inducible promoters from Streptomyces lividans, P_{BAD}, an araC gene from E. coli; P_{bla} a blal gene, from Staphylococcus aureus; P_{gal} a galR gene from Streptococcus mutans, Pₘₐₗ, and a mulR gene from Streptococcus pneumoniae.

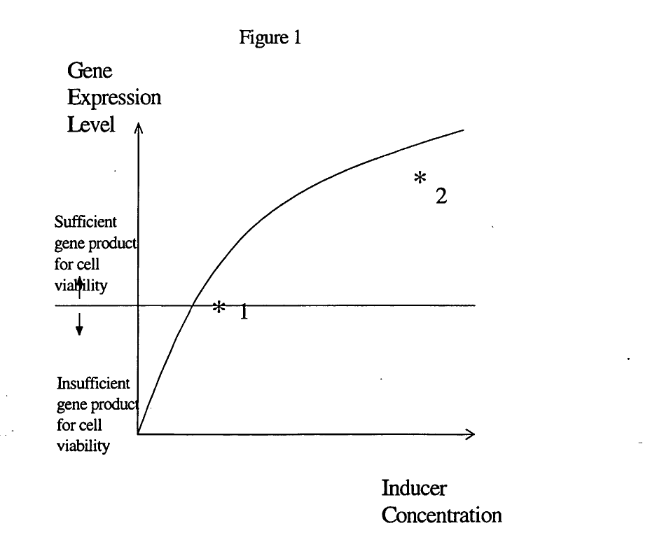

The levels of a gene product can be titrated by varying the level of an inducer for any given inducible promoter (e.g., xylose for P_{xylA}; IPTG for P_{spac}, etc.). In a preferred embodiment, a therapeutic compound screen may be run at both level 1 and level 2 (see FIG. 1), or at additional levels, and therapeutic compound hits are determined, for example, by their ability to reduce host cell viability or growth or induce lysis, as measured by any method known in the art to detect such changes in the state of a host cell, such as, a reduction in the rate of increase of optical density (herein “OD”) at 600 nm, or other appropriate OD or detection method, at level 1 but not level 2, or some other level. Such hits are deemed to act specifically on the gene product, on the basis that they are not potent enough to inhibit all of the excess of gene product present at level 2, and that, in the case of antimicrobial compounds, general antimicrobials will inhibit at both levels, and at other levels. However, not all hits that inhibit at both levels, or other levels, will, in the case of antimicrobial compounds, be general antimicrobials. Gene product-specific inhibitors that are particularly potent work at both levels. Therefore, a further screen may be employed for hits in that category. This involves rerunning the screen using a reduced concentration of these hit compounds, to look for any that only cause a reduction in host cell viability at level 1. These are deemed to be gene product-specific inhibitors. Therapeutic compound hits are also determined, for example, by their ability to increase or enhance host cell viability, growth, proliferation or differentiation, as measured by any method known in the art to detect such changes in the state of a host cell, such as, a increase in the rate of increase of OD at 600 nm, or other appropriate OD or detection method, at level 1 but not level 2, or some other level.

As used herein “altered metabolism” means any detectable change in a host cell, such as, an alteration or modulation in viability, growth, proliferation, differentiation, gene expression, gene product activity, lysis, cell division, chemotaxis, motility, cytoskelatel structure or motion, nuclear structure, meiosis, mitosis, translation, transcription, sister chromatid exchange, cell permeability, surface receptors, refractive index, sporulation, tumbling in solution, optical density, protein folding, protein content, nucleic acid content, phagocytosis, or protein stability. The skilled artisan can readily determine which of these states of altered metabolism are relevant to prokaryotes and/or eukaryotes.

An alternative preferred embodiment of this screen invention uses a different, preferably more sensitive readout to OD alteration in order to assess therapeutic compounds. This involves co-expressing a reporter molecule, such as green fluorescent protein or luciferase, among other marker genes and gene products well known in the art. Therapeutic compound hits are identified by their ability to reduce the reporter output, such as fluorescence or luminescence, among other marker genes and gene products well known in the art.

Following contacting the host cell with at least one candidate compound, if it is determined that there is reduced host cell viability, the candidate compound may be useful as a therapeutic compound. This may be readily determined using any of the many well known methods for testing therapeutic activity, particularly antimicrobial activity, such as, for example, by disk diffusion assay followed by an MIC determination.

Another application of this technology is for determining the cellular target of therapeutic compounds. The natural promoter of the proposed target gene is replaced with a heterologous, regulatable promoter in the chromosome of a gene-expressing host cell, particularly a microbial host cell, as described above. Host cells are grown in the presence of varying amounts of inducer and therapeutic compound. If the level of host cell viability, as measured for example by OD or reporter levels, is directly proportional to the levels of inducer, then the compound is deemed to act specifically against the target gene or gene product. Two alternative methods, among others, for varying a cellular level of the target protein are: 1) increasing target protein level by transforming a host cell or cells with a plasmid containing an additional copy of the target gene under an inducible promoter or 2) decreasing a target protein level by transforming a cell or cells with a plasmid containing a partial antisense strand of a target gene.

It is preferred that the method of the invention is formatted for high throughput screening (herein “HTS”). Skilled artisans can readily adapt the method of the invention for HTS. A particularly preferred embodiment of the screening methods of the invention is a high throughput screen for compounds that interfere with the proper functioning of gene expression or protein.

Potential therapeutic compounds identified using the method of the invention include, among other things, small organic molecules, polynucleotides, peptides, polypeptides and antibodies that bind host cell polynucleotides or polypeptides, or mimic the activity of a host cell polypeptides.

Potential antagonists include a small molecule that binds to a host cell polynucleotides or polypeptides thereby preventing binding of natural factors such that normal biological activity is prevented. Examples of small molecules include but are not limited to small organic molecules, peptides or peptide-like molecules. Small molecules of the invention preferably have a molecular weight below 2,000 daltons, more preferably between 300 and 1,000 daltons, and most preferably between 400 and 700 daltons. It is preferred that these small molecules are organic molecules. Any molecule from any source can be used as a candidate compound in the methods of the invention, but it is preferred that candidate compounds be small organic molecules.

The invention further provides assay packs and kits comprising one or more containers filled with one or more of the ingredients of the aforementioned compositions of the invention. Examples of preferred kits are kits comprising at least one host cell lacking a host cell gene and the host cell comprising at least one recombinant, regulatable host cell gene of the invention. A further preferred kit comprises a polynucleotide encoding a recombinant host cell gene controlled by an regulatable promoter. Kits comprising a host cell gene expressibly linked to an regulatable promoter are also preferred.

### EXAMPLES

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail.

All parts or amounts set out in the following examples are by weight, unless otherwise specified.

### Example 1

### Development of a RAT Gene-Based Whole Cell Assay in S. aureus for Discovery of Antimicrobial Compounds

The RAT operon (ratC-ratA-ratB) encodes an essential, heterotrimeric protein called tRNA-dependent amidotransferase (hence RAT; also known as Glu-tRNA^{Gln} amidotransferase or Glu-AdT—Cumow-A W, et al. PNAS 94, 11819-11826 (1997)). The natural promoter of the RAT operon was replaced with a heterologous, regulatable promoter (Pspac) plus a constitutively expressed lad gene, in the chromosome of S. aureus RN4220 by insertional mutagenesis. In this way, there is an extra copy of the first stretch of 500 base pairs of the RAT operon present, still under the control of the native promoter. This is not sufficient sequence to encode active RAT protein.

The levels of full length RAT protein could be titrated by varying the level of IPTG inducer between 0 and 1 mM, showing the same curve as in FIG. 1. Immunoblot experiments using a RAT-specific polyclonal antibody demonstrated that, even in the absence of inducer, the space promoter was leaky, such that RAT was expressed above wild type levels. In order to reduce the leakiness of the space promoter, and to identify the minimal IPTG level for cell viability, a LacI overexpression plasmid (pLacI) was transformed into the recombinant strain. Immunoblot experiments on this new strain demonstrated that the RAT protein was still titratable by varying the level of IPTG, at uniformly lower levels than in the strain lacking pLacI. From these experiments it was possible to identify IPTG levels corresponding to RAT levels 1 and 2, as depicted in FIG. 1 and suitable for use in the antimicrobial screen of this invention.

### Example 2

### Determination of Cellular Target of a Compound Using a S. aureus Strain with Regulatable def1 Gene

The def1 gene in Staphylococcus aureus encodes an essential protein called polypeptide deformylase. The natural promoter of the def1 single gene operon was replaced with a heterologous, regulatable promoter (Pspac) plus a constitutively expressed lacI gene, in the chromosome of S. aureus RN4220 by insertional mutagenesis. In this way, there is an extra copy of the first stretch of 450 base pairs of the def1 gene present, still under the control of the native promoter. This is not sufficient sequence to encode functional Def1 protein due to the absence of conserved metal-binding amino acids at the C-terminal of the protein.

The levels of Def1 protein could be titrated by varying the level of IPTG inducer between 0 and 1 mM, showing the same curve as in FIG. 1. The antimicrobial activity of an inhibitor compound of Def1, determined through high throughput screening using an in vitro enzymatic assay, was tested against the regulatable def1 strain at various IPTG concentrations. Increasing concentrations of the inducer, which led to increasing amount of Def1 protein in the cell, resulted in elevation of the MIC values of the compound (Table 1). A control experiment using nine antibiotics nonspecific to Def1 protein did not show variation of MIC values under different inducer concentration. Therefore, the antimicrobial activity of the compound is due to the specific inhibition of the polypeptide deformylase. This experiment demonstrates an application of the invention using strains with regulatable promoter to identify the cellular targets of antimicrobial compounds.

TABLE 1
Minimum Inhibitory Concentration of
SB-220334 on S.aureus RN4220/def1 strain

| | | | | | | | | |
|---|---|---|---|---|---|---|---|---|
| [IPTG] μg/ml | 0 | 1.5 | 3.125 | 6.25 | 12.5 | 25 | 50 | 100 |
| MIC μg/ml | 0.5 | 1 | 4 | 8 | 32 | >32 | >32 | >32 |

### Example 3

### Downregulation of a Target Gene Expression in S. aureus Using a Regulated Antisense hla Gene

The hla gene encodes alpha-toxin in S. aureus. A 621 bp hla fragment was cloned into pYJ335 downstream of the tetracycline inducible promoter (Ptet/xyl) in antisense orientation. This shuttle vector carrying antisense hla construct was introduced into S. aureus WCUH29.

The expression of alpha-toxin could be downregulated after induction transcription of antisense hla RNA using tetracycline or anhydrotetracycline. Induced antisense hla RNA downregulated chromosomally derived hla gene expression in vitro approximately 14-fold. Most importantly, this reduction completely eliminated the lethality of the infection. In contrast, a control S. aureus carrying sense hla construct did not show any effect on expression of alpha-toxin in vitro and on the lethality of the infection at the presence of induction. These results indicate that elimination of toxicity of S. aureus is due to the specific down-regulation of expression of alpha-toxin. Moreover, this inducible antisense system is titratable by varying the level of inducer. The titatability of this promoter system makes it possible to evaluate the effects of different levels of downregulation of an essential target gene either in culture condition or in an animal model of infection without completely inactivating it in turn can aid in the development of antimicrobial agents by decreasing levels of a target gene product and potentially rendering cells more susceptible.

Each reference disclosed herein is incorporated by reference herein in its entirety. Any patent application to which this application claims priority is also incorporated by reference herein in its entirety.

What is claimed is:

1. A method for identifying candidate compounds which alter metabolism comprising the steps of: providing at least one host cell comprising at least one recombinant regulatable gene; contacting said host cell with at least one candidate compound at least one level of induction of gene expression; and detecting altered metabolism in said host cell of the contacting step.

2. The method of claim 1 wherein the recombinant gene is on an episomal element or integrated into a chromosome of said host cell.

3. The method of claim 1 wherein the at least one level is two or more levels.

4. The method of claim 1 wherein said at least one recombinant regulatable gene is from S. aureus.

5. The method of claim 1 wherein said altered metabolism comprises an alteration or modulation in growth.

6. The method of claim 1 wherein said detecting step comprises detecting an optical density.

7. A method for identifying candidate compounds which alter metabolism comprising the steps of: providing at least one host cell comprising at least one recombinant regulatable gene; contacting said host cell in a first contacting step with at least one candidate compound at a first level of induction of gene expression; detecting altered metabolism in said host cell of said first contacting step; contacting a second host cell or said host cell in a second contacting step with at least one candidate compound at a second level of induction of gene expression;
and detecting altered metabolism in said host cell of said second contacting step.

8. The method of claim 7 wherein the recombinant gene is on an episomal element or integrated into a chromosome of said host cell.

9. The method of claim 7 wherein said at least one recombinant regulatable gene is from S. aureus.

10. The method of claim 7 wherein said altered metabolism comprises an alteration or modulation in growth.

11. The method of claim 7 wherein said detecting step comprises detecting an optical density.
